(19) United States
(12) Patent Application Publication
(10) Pub. No.: US 2010/0010596 A1
(43) Pub. Date: Jan. 14, 2010

(54) METHOD AND SYSTEM RELATING TO HYPERTHERMIA

(75) Inventors: Mikael Persson, Alingsas (SE); Hana Trefna, Vastra Frolunda (SE)

Correspondence Address: YOUNG & THOMPSON, 209 Madison Street, Suite 500, Alexandria, VA, 22314, US

(73) Assignee: ELECTROMAGNETIC CONSULTING SWEDEN AB, Arlingsas (SE)

(21) Appl. No.: 12/302,119

(22) PCT Filed: May 23, 2007
(86) PCT No.: PCT/SE07/00502

§ 371 Date: Mar. 23, 2009

Related U.S. Application Data
(60) Provisional application No. 60/747,939, filed on May 23, 2006.

(51) Int. Cl. A61B 18/04

(52) U.S. Cl. 607/100

(57) ABSTRACT

A method and system for selective heating of an object based on a model of the object, the method comprising the steps of: modeling of a wave front of a source, which wave front is propagated through the model object from a virtual antenna placed in the model of the specific region where heating is desired, simulating a radiated field and measuring the same using computer models of surrounding antenna system, time-reversing, transferring and synthesizing the signal in a real system, transmitting by a real antenna system the field in a time reversed order, and refocusing of time reversed signal at the original s the invariance of a wave equation under time reversal.

### TECHNICAL FIELD OF THE INVENTION

The present invention relates to a method and system for selective heating of an object based on a model of the object.

### BACKGROUND OF THE INVENTION

Hyperthermia (or microwave thermotherapy) is presently used as an adjuvant to the radiation therapy in the treatment of certain types of cancers [1]-[6]. The goal of hyperthermia treatment is to raise the temperature of a localized cancerous tumor to a therapeutic level without overheating surrounding normal tissue. The effective temperature range of hyperthermia is normally 39 C-44 C. The power deposition within a patient is governed by the interaction of the irradiated electric field with patient's tissues. The interaction is rather complex due to the non homogeneous dielectric properties of the tissues. It is further complicated by the cooling produced significantly varying blood perfusions within the heated volume. One approach to provide deep hyperthermia is to use an array of radiators placed in a circumferential array around the patient, relying on constructive wave interference to selectively heat the tumor, [4]-[6]. However, until now, no efficient method has been available to selectively heat the deep-seated tumors resulting in undesired, limiting heating of and hotspots in surrounding tissues. The importance of preventing hot spots, is indicated by the observation that treatment-limiting hot spots occur in more then 80% of all regional hyperthermia patients [7]. The novel concept of this application based on time reversal near field beam forming solves this problem.

### SHORT DESCRIPTION OF THE INVENTION

The objective of this invention is to provide a method and arrangements for selective heating of specific part of objects without undesired heating of surrounding parts of the object.

This objective has been achieved by a coupling between, the mathematical model of the system, including the object heated, and the real antenna system, including the real object heated. Through this coupling information of the influence of the object on the wave, obtained in the model system is used in the real system through the time reversal characteristics of the wave field to focus the deposited energy of the field to a to predetermined region.

According to a first aspect of the invention, a method for selective heating of an object based on a model of the object is provided. The method comprises the steps of:
modeling of a wave front of a source, which wave front is propagated through the model object from a virtual antenna placed in the model of the specific region where heating is desired,
simulating a radiated field and measuring the same using computer models of surrounding antenna system,
time-reversing, transferring and synthesizing the signal in a real system,
transmitting by a real antenna system the field in a time reversed order, and
refocusing of time reversed signal at the original s the invariance of a wave equation under time reversal.

The method is further characterized by using double or multiple time reversal for applications, in particular for, but not limited to, cases were access is limited.

According to a second aspect of the invention a system is provided for selective heating of an object based on a model of the object using wave front of a source propagated through an object from a virtual antenna placed in a section of object specific dielectric model. The system comprises a modeling portion and a real portion, the modeling portion comprising a computer unit for simulation of a radiated field and virtually measuring the field using a computer model of a surrounding antenna system, the real portion comprising a real antenna system for transmitting the field in a time reversed order, means for utilizing the time reversal characteristics of waves to focus an intensity to a specified region, a model detection system for detecting radiation of the model system by a model surrounding the region of focusing, means for reradiating theoretically detected field using a true implementation of the system using the time reversal characteristics focusing the intensity of the fields in the desired region. Most preferably, the waves are electromagnetic waves or sound waves. The invention can be used for medical hyperthermia for cancer treatment or for other medical treatment. It is possible to use double or multiple time reversal for applications, in particular for, but not limited to, cases were access is limited. The system may have means for using information on the location of the tumor as obtained from the same or other, microwave, ultrasound or other system or other image generating systems, which could be but is not limited to CT or MRI. The system can be used for treatment on breast cancer and other cancer forms. The system further comprises a signal generator for generating a signal, an amplifier for amplifying the signal from the signal generator, which minimizes system noise figure and provide enough gain with sufficient linearity, a Power Divider Network for dividing the signal into N paths, an attenuator, which reduce reflections in the divided signal, phase shifter for phase shifting and amplifier for amplifying each path according to values obtained in the simulation part and sent the signals to the real antenna. In the system the power amplifier and antenna are connected by a matching network and circulator, to protect the system against reflected waves and to match the characteristic impedance.

### SHORT DESCRIPTION OF THE DRAWINGS

In the following the invention is described with reference to an exemplary embodiment illustrated in the attached drawings, in which:

FIG. 1 illustrates simulated normalized power distributions calculated in a neck slice containing tumors of different sizes.

FIG. 2 shows the simulated average absorption ratio values as a function of number of antennas for different volume sizes for the same neck case as in FIG. 1.

FIG. 3 shows the average absorption ratio values as a function of frequency for different volume sizes for a breast model slice as shown in FIG. 4.

FIG. 4 shows a breast model slice and the simulated normalized power distributions calculated in this model containing a tumors of 5 mm radius.

FIG. 5 shows the schematic of the time reversal beam former hyperthermia system.

### DETAILED DESCRIPTION OF THE EMBODIMENTS

The basic principle of the new method and device is the coupling between the electromagnetic modeling of the system and the real antenna system. In the complete system the modeling of the wave front of the source is propagated through the model object from a virtual antenna placed in the model of the specific region where heating is desired. The simulated radiated field is then measured, virtually, using the computer models of the surrounding antenna system. The signal is the time-reversed, transferred and synthesized in the real system. The real antenna system is then transmitting this field in a time reversed order. It is the invariance of the wave equation under time reversal which enables optimal refocusing of time reversed signal at the original source. While not perfect in lossy media the method has, as indicated in the figures been proven to be efficient for lossy cases as well.

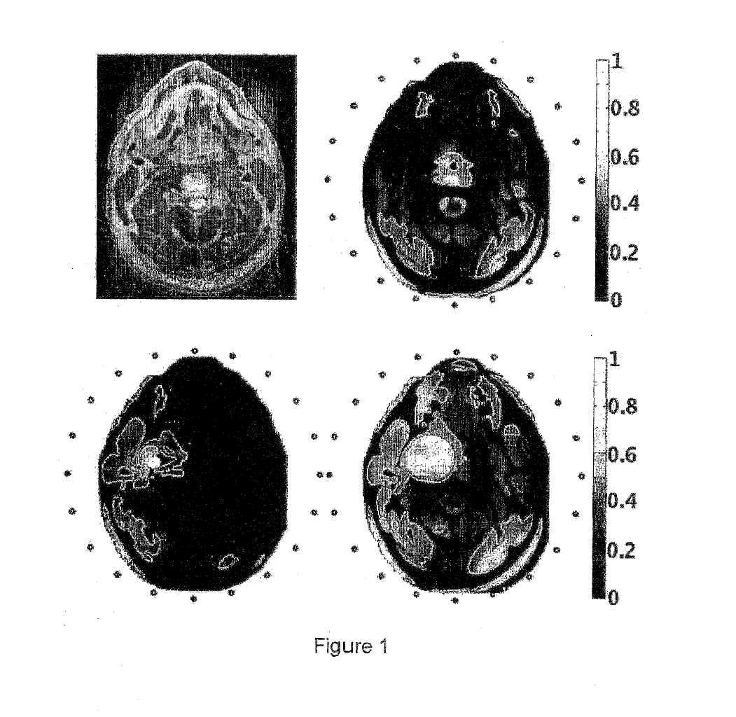

FIG. 1 shows a neck slice and normalized absorbed power distribution calculated in a neck model containing tumour with radii of for example 5, 5 and 23 mm radius. The frequency is 500 Mhz.

In FIG. 1 the simulation of the embodied system shows a considerable heating, in terms of the specific power absorption of a predetermined region without considerable heating of the surrounding region. A tumour like object is placed in the vicinity of spinal cord in the centre of the neck. The obtained absorbed power distribution is very favourable, although high level of energy is absorbed on the surface of the body. It is apparent that the increased power on the skin is local and not expected to cause problems as it will be cooled by blood perfusion and by water bolus.

The system performance can be expressed in terms of the aPA ratio

$$aPA = \frac{{\sum }^{{V}_{tum}} \frac{PA}{{N}_{{V}_{tum}}}}{{\sum }^{{V}_{rt}} \frac{PA}{{N}_{{V}_{rt}}}} \qquad ( 1 )$$

Which indicate two major aspects of the applicator performance: selective heating of the desired region and the ability of applicator to avoid hot spots in undesired regions. A good applicator should have a high aPA. The sum in the denominator and nominator of Eq. 1 represent a summation over the tumour tissue volume Vₜᵤₘ and the corresponding elements of the non-tumour tissue volume Vᵣₜ respectively. Nₜᵤₘ and Nᵥᵣₜ are the total number of volume elements of the tumour tissue and the total number of volume of the non-tumour tissues respectively. PA is the specific power absorption

$$PA = \frac{σ {\vert \vec{E} \vert}^{2}}{2} [ \frac{W}{{m}^{3}} ] \qquad ( 2 )$$

Where σ [S/m] is the electrical conductivity of the tissue and |E| [V/m] is the magnitude of electric field.

The initial temperature rise ΔT [° C.] in tissue, disregarding cooling is related to the absorbed power PA as given in Eq. 3.

$$Δ T = \frac{1}{c} PA \cdot Δ t [ ° C . ] \qquad ( 3 )$$

where Δt is the time period of exposure in units seconds and c [J/(kg° C.)] is the specific heat capacity of tissue.

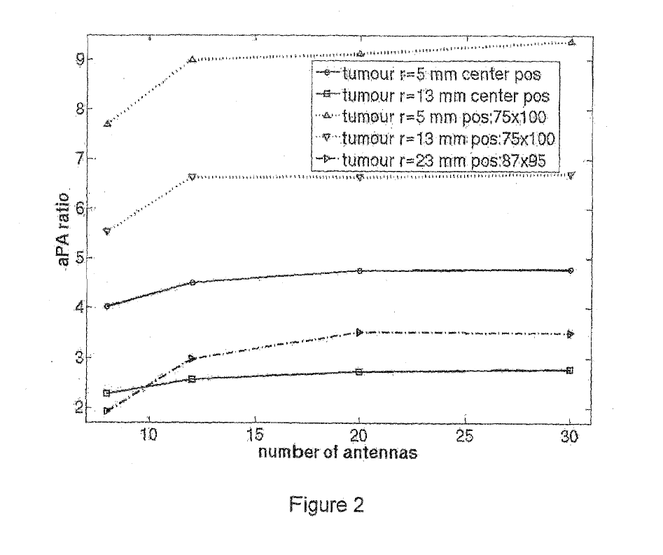

FIG. 2 shows the simulated average absorption ratio values, aPA, as a function of number of antennas for different volume sizes for the same neck case as in FIG. 1. We see that the highest aPA ratio was obtained with 30 radiators, but the result starts to plateau at 12 radiators which thus gives a good cost benefit ratio. The study was performed using the frequency 500 MHz. This frequency represents a good compromise between penetration depth and focusing ability of the algorithm. The results demonstrate the feasibility of the embodied design.

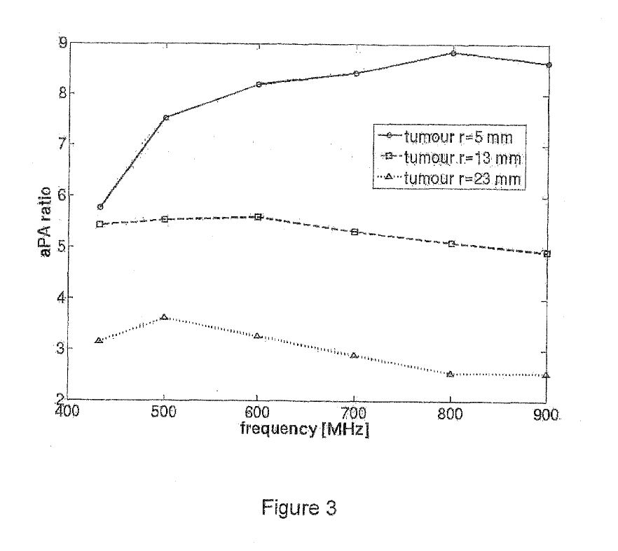

In a separate series of simulations using a breast model the influence of the frequency on the ability to selectively heat tumours of different sizes is tested. The results, summarized in FIG. 3, show the aPA ratio for different tumour sizes and locations versus the frequency of the electromagnetic field. The system focuses electromagnetic energy better in higher frequencies. The average power absorption (aPA) ratio as a function of frequency for different target volume sizes.

Hence, these frequencies are more suitable for treatment of the small tumours while for large tumour strong focusing is not such an advantage as we attempt for a homogeneous heating of the whole tumour volume. The importance of preventing hot spots, which is here claimed to be related to high PA maxima in undesired regions and low aPA occur in more then 80% of all regional hyperthermia patients [7]. The results with high aPA demonstrates the feasibility of the embodied design.

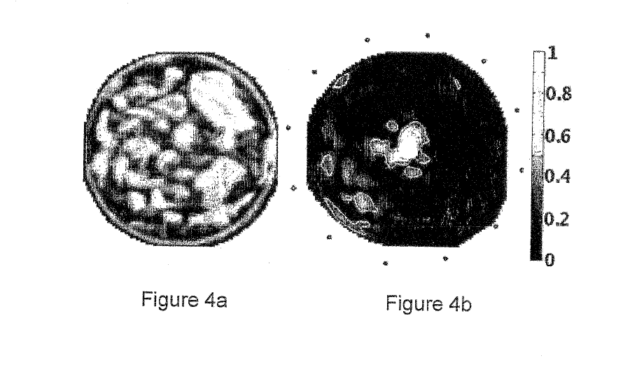

FIG. 4 revels the capability of the proposed system to focus energy in tumours in a breast model. Due to the smaller dimensions than for the neck case showed previously, hyperthermia treatment of this region is easier. In FIGS. 4a and 4b normalized absorbed power distribution calculated in a breast containing tumour of radius 5 mm, situated in the centre of the breast using the frequency 800 Mhz. The results demonstrate the feasibility of the embodied design.

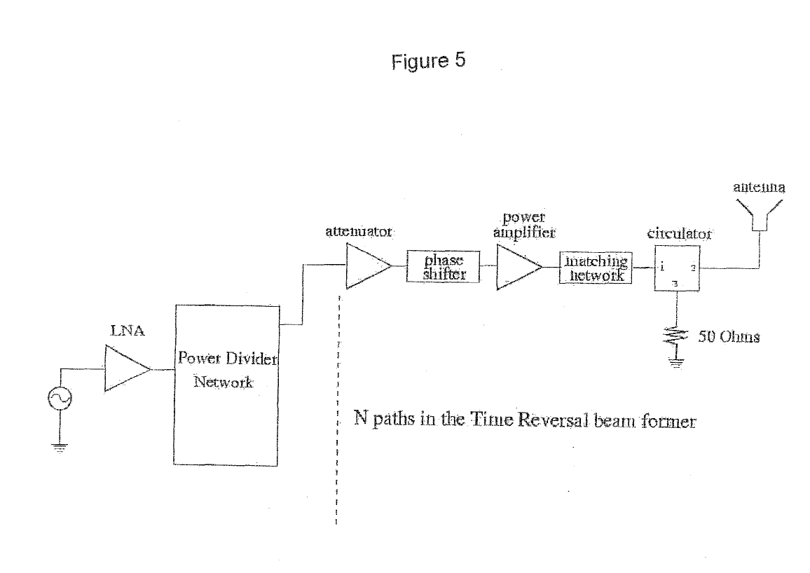

FIG. 5 shows a block diagram of an embodied system. A more detailed schematic of the block diagram shown in FIG. 5 is presented. The signal generated by the generator is amplified by low noise amplifier, which minimizes the system noise figure and provide enough gain with sufficient linearity. The signal is then divided to N paths by the Power Divider Network. The signal then goes trough attenuator, which reduce reflections. The signal in each path is then phase shifted and amplified according to values obtained in simulation part and sent to the antenna. The power amplifier and antenna are connected by matching network and circulator, to protect the system against reflected waves and to match the characteristic impedance to 50 Ohms. The embodied system uses the time reversal algorithm as it delivers a very accurate estimate for the relative amplitude and phase for each sensor compared to the central element to achieve field focusing at a desired point. Once the relative amplitude and phase is obtained, it can then be programmed in to each amplitude and phase control unit of the antenna array.

Most preferably, the waves are electromagnetic waves or sound waves. The invention can be used for medical hyperthermia for cancer treatment or for other medical treatment. It is possible to use double or multiple time reversal for applications, in particular for, but not limited to, cases were access is limited. The system may have means for using information on the location of the tumor as obtained from the same or other, microwave, ultrasound or other system or other image generating systems, which could be but is not limited to CT or MRI. The system can be used for treatment on breast cancer and other cancer forms.

### REFERENCES

[1] G. Hahn: Hyperthermia and Cancer. New York: Plenum, 1982.
[2] J. VanderZee, D. G. Gonzales, G. C. VanRhoon, J. D. P. VanDijk, W. L. J. VanPutten, and A. A: M. Hart: Comparison of radiotherapy alone with radioterapy plus hyperthermia in locally advanced pelvic tumors: A Prospective, randomised, multicentre trial, Lancet, vol. 355, pp. 1119-1125, 2000.
[3] P. F. Turner: Regional hyperthermia with an annular phased array. IEEE Trans. Biomed. Eng., vol. BME-31, pp. 106-114, 1984.
[4] P. F. Turner: Hyperthermia and inhomogeneous tissue effects using an annular phased array. IEEE Trans. Microwave Theory Tech., vol. MTT-32, pp. 874-882, 1984.
[5] M. E. Kowalski, J-M. Jin: Model-Based Optimalization of Phased Arrays for Electromagnetic Hyperthermia. IEEE Trans. Microwave Theory Tech., vol. 52, no. 8, pp. 1964-1977, August 2004.
[6] J. Nadobny, W. Wlodarczyk, et al.: A Clinical Water-Coated Antenna Applicator for MRControlled Deep-Body Hyperthermia: A Comparison of Calculated and Measured 3-D Temperature Data Sets. IEEE Trans. Biomed. Eng., vol. 52, no. 3, pp. 505-519, March 2005.
[7] P. Wust, J. Nadobny, M. Seebass, D. Stalling, J. Gellermann, H. C. Hege, P. Deuflhard and R. Felix, “Influence of patient models and numerical methods on predicted power deposition patterns, Int. J. Hyperth, vol. 15, pp. 519-540, 1999.

1. A method for selective heating of an object based on a model of said object, the method comprising the steps of:
modeling of a wave front of a source, which wave front is propagated through the model object from a virtual antenna placed in the model of the specific region where heating is desired,
simulating a radiated field and measuring the same using computer models of surrounding antenna system,
time-reversing, transferring and synthesizing the signal in a real system,
transmitting by a real antenna system the field in a time reversed order, and
refocusing of time reversed signal at the original s the invariance of a wave equation under time reversal.

2. The method according to claim 1, characterized by using double or multiple time reversal for applications, in particular for, but not limited to, cases were access is limited.

3. A system for selective heating of an object based on a model of said object using wave front of a source propagated through an object from a virtual antenna placed in a section of object specific dielectric model, characterized in that the system comprises a modeling portion and a real portion, said modeling portion comprising a computer unit for simulation of a radiated field and virtually measuring said field using a computer model of a surrounding antenna system, the real portion comprising a real antenna system for transmitting the field in a time reversed order, means for utilizing the time reversal characteristics of waves to focus an intensity to a specified region, a model detection system for detecting radiation of the model system by a model surrounding the region of focusing, means for reradiating theoretically detected field using a true implementation of the system using said time reversal characteristics focusing the intensity of the fields in the desired region.

4. The system according to claim 3, wherein said waves are electromagnetic waves or sound waves

5. The system according to claims 3, used for medical hyperthermia for cancer treatment or for other medical treatment.

6. The system according to claim 3, characterized by using double or multiple time reversal for applications, in particular for, but not limited to, cases were access is limited.

7. A system according to claim 3, characterized by having means for using information on the location of the tumor as obtained from the same or other, microwave, ultrasound or other system or other image generating systems, which could be but is not limited to CT or MRI.

8. The system according to claim 3, applied to treatment on breast cancer and other cancer forms.

9. The system according to claim 3, further comprising a signal generator for generating a signal, an amplifier for amplifying said signal from said signal generator, which minimizes system noise figure and provide enough gain with sufficient linearity, a Power Divider Network for dividing said signal into N paths, an attenuator, which reduce reflections in said divided signal, phase shifter for phase shifting and amplifier for amplifying each path according to values obtained in said simulation part and sent said signals to said real antenna.

10. The system of claim 9, wherein said power amplifier and antenna are connected by a matching network and circulator, to protect the system against reflected waves and to match the characteristic impedance.

11. The system according to claims 4, used for medical hyperthermia for cancer treatment or for other medical treatment.

12. The system according to claim 4, applied to treatment on breast cancer and other cancer forms.

13. The system according to claim 5, applied to treatment on breast cancer and other cancer forms.

14. The system according to claim 6, applied to treatment on breast cancer and other cancer forms.

15. The system according to claim 7, applied to treatment on breast cancer and other cancer forms.

16. The system according to claim 4, further comprising a signal generator for generating a signal, an amplifier for amplifying said signal from said signal generator, which minimizes system noise figure and provide enough gain with sufficient linearity, a Power Divider Network for dividing said signal into N paths, an attenuator, which reduce reflections in said divided signal, phase shifter for phase shifting and amplifier for amplifying each path according to values obtained in said simulation part and sent said signals to said real antenna.

17. The system according to claim 5, further comprising a signal generator for generating a signal, an amplifier for amplifying said signal from said signal generator, which minimizes system noise figure and provide enough gain with sufficient linearity, a Power Divider Network for dividing said signal into N paths, an attenuator, which reduce reflections in said divided signal, phase shifter for phase shifting and amplifier for amplifying each path according to values obtained in said simulation part and sent said signals to said real antenna.

18. The system according to claim 6, further comprising a signal generator for generating a signal, an amplifier for amplifying said signal from said signal generator, which minimizes system noise figure and provide enough gain with sufficient linearity, a Power Divider Network for dividing said signal into N paths, an attenuator, which reduce reflections in said divided signal, phase shifter for phase shifting and amplifier for amplifying each path according to values obtained in said simulation part and sent said signals to said real antenna.

19. The system according to claim 7, further comprising a signal generator for generating a signal, an amplifier for amplifying said signal from said signal generator, which minimizes system noise figure and provide enough gain with sufficient linearity, a Power Divider Network for dividing said signal into N paths, an attenuator, which reduce reflections in said divided signal, phase shifter for phase shifting and amplifier for amplifying each path according to values obtained in said simulation part and sent said signals to said real antenna.
